(19) United States
(12) Patent Application Publication
(10) Pub. No.: US 2021/0257096 A1
(43) Pub. Date: Aug. 19, 2021

(54) INFLUENCING END-STAGE RENAL DISEASE OUTCOMES THROUGH PREDICTING PHYSIOLOGICAL PARAMETERS AND DETERMINING DOSING RECOMMENDATIONS

(71) Applicant: University of Virginia Patent Foundation, Charlottesville, VA (US)

(72) Inventors: Donald E. Brown, Charlottesville, VA (US); Brendan T. Bowman, Charlottesville, VA (US); Benjamin J. Lobo, Charlottesville, VA (US)

(21) Appl. No.: 17/180,301

(22) Filed: Feb. 19, 2021

Related U.S. Application Data
(60) Provisional application No. 63/066,960, filed on Aug. 18, 2020.
(60) Provisional application No. 62/978,687, filed on Feb. 19, 2020.

(51) Int. Cl. G16H 50/20; A61B 5/00; G16H 50/70; G16H 70/40; G16H 10/60; G16H 50/30; G16H 15/00; G16H 20/13; G06N 3/04

(52) U.S. Cl. CPC G16H 50/20; A61B 5/7267; A61B 5/7275; G16H 50/70; G16H 70/40; A61B 5/14546; G16H 50/30; G16H 15/00; G16H 20/13; G06N 3/0454; G06N 3/0445; G16H 10/60

(57) ABSTRACT

In some aspects, a system is disclosed for predicting values of a physiological parameter for a patient. The system can include a processor and a memory device. The processor can: receive measurements of a physiological parameter for a patient; determine, using a recurrent neural network, values of the physiological parameter from the measurements for the patient; and output the values for presentation to a caregiver. The memory device can store the recurrent neural network.

### CROSS-REFERENCE TO RELATED APPLICATIONS

This application claims priority to U.S. Patent Application Nos. 62/978,687 and 63/066,960 respectively filed on Feb. 19, 2020, and Aug. 18, 2020; the disclosures of which are hereby incorporated by reference in their entirety.

### BACKGROUND

Embodiments of the present disclosure relate to apparatuses, systems, and methods for influencing end-stage renal disease outcomes.

### BRIEF DESCRIPTION OF THE DRAWINGS

Embodiments of the present disclosure will now be described hereinafter, by way of example only, with reference to the accompanying drawings in which:

FIG. 1 illustrates an example computing environment;

FIG. 2 illustrates example prediction model usable within the computing environment of FIG. 1;

FIG. 3 illustrates an example process performable within the computing environment of FIG. 1;

FIG. 4 illustrates an example machine usable to construct one or more of the devices or systems within the computing environment of FIG. 1; and

FIG. 5 illustrates an example computer system usable to construct one or more of the devices or systems within the computing environment of FIG. 1.

### DETAILED DESCRIPTION

### Overview

This application describes approaches for improving end-stage renal disease (ESRD) outcomes through predicting values of one or more physiological parameters of a patient and providing dosing recommendations based on the predicted values. The predicted values can be determined using one or more recurrent neural networks (RNNs). Inputs to one or more of the recurrent neural networks can include one or more of (i) measured values of the one or more physiological parameters for the patient, (ii) past dosing amounts or timings for the patient, (iii) laboratory test data for the patient, (iv) dialysis data for the patient, or (v) planned or potential future dosing amounts or timings for the patient. The physiological parameters for which values are predicted can include hemoglobin (Hgb) or parathyroid hormone (PTH), among other possibilities. Where the physiological parameter for which values are predicted is hemoglobin, the dosing recommendations can include an indication of an amount or a timing of erythropoesis stimulating agent (ESA) to be dosed to the patient. Where the physiological parameter for which values are predicted is parathyroid hormone, the dosing recommendations can include an indication of an amount or timing of a calcimimetic drug, such as etelcalcetide, to be dosed to the patient.

One or more approaches described herein can facilitate a dose optimized so that a minimum amount of a drug (sometimes referred to as a compound) may be used to achieve a desired clinical outcome. This can limit side-effects that may be experienced by a patient from dosing of the drug, as well as a cost of the drug that is dosed. Such an optimization may also be desirable because the effect of a change in dosing of the drug may take multiple months (for instance, 2 or 3 months) to be realized in the patient while dosing decisions may be made by a clinician monthly, and the time delayed effect of previous doses on the physiological parameter of the patient may be difficult for the clinician to anticipate.

One or more approaches described herein can determine predicted values (for instance, levels) of a physiological parameter of a patient and provide dosing recommendations for the patient from the predicted values. As a result, dosing and patient care may be customized to the patient and the patient's anticipated individual response to the dosing. This may desirably prevent physiological parameter cycling, such as hemoglobin cycling, where the values of the physiological parameter cycle about a particular level, such as a target level.

One or more approaches described herein can determine predicted values of a physiological parameter under multiple different dosing regimens. This can enable a clinician to perform a what-if analysis to better understand the impact of different selected amounts or timings of drug dosing for a patient. The clinician may thus select from the multiple different dosing regimens a particular dosing regimen that achieves a desired clinical outcome for the patient.

### Dosing Management

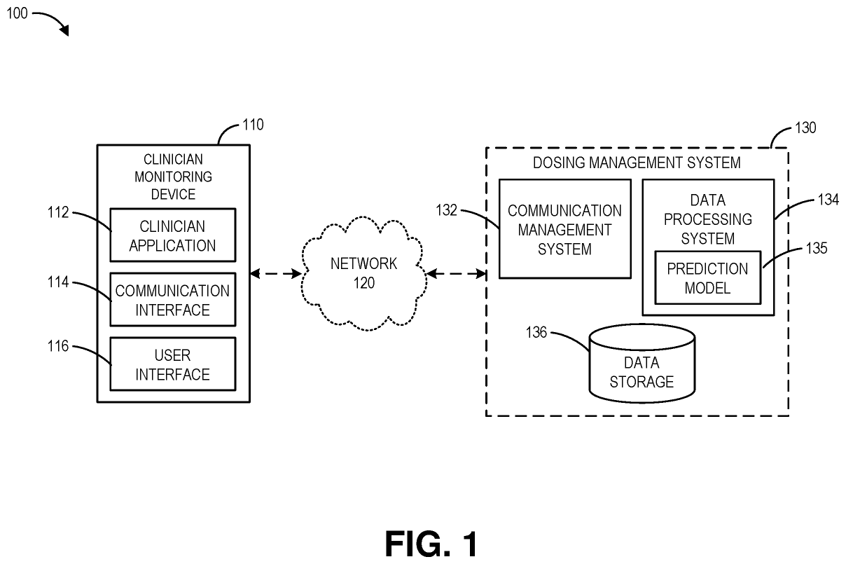

FIG. 1 illustrates a computing environment 100 for dosing management. The computing environment 100 includes a clinician monitoring device 110 in communication with a monitoring management system 130 via a network 120.

The clinician monitoring device 110 can be operated by a clinician, such as an individual who supervises, assists, or cares for a patient. The clinician monitoring device 110 may be a computing device such as a smart phone, a tablet computer, or a desktop computer. The clinician monitoring device 110 can include a clinician application 112, a communication interface 114, and a user interface 116.

The clinician application 112 can be program that is executed by processor of the clinician monitoring device 110. The clinician application 112 can enable the clinician monitoring device 110 to communicate via the communication interface 114 with the dosing management system 130. The clinician application 112 may (i) collect, process, review, present, or transmit measured data or predicted data about the patient and (ii) allow the clinician to generate, review, or adjust dosing recommendations that are prepared by the dosing management system 130 or analyze other data stored or monitored by the dosing management system 130. The clinician application 112 can present data or information to the clinician via one or more graphical user interfaces of the user interface 116, such as on a display or a touchscreen. The clinician application 112 may, for example, receive planned or potential future dosing regimens for a patient or present dosing recommendations for the patient determined by the dosing management system 130. The clinician application 112 may function, in combination with the dosing management system 130, as a decision support tool to assist the clinician in making dosing decisions for the patient.

The dosing management system 130 can be a computing device and include a communication management system 132, a data processing system 134, and a data storage 136 that may be in communication with one another. The dosing management system 130 may, for instance, be constructed partly or entirely of a server infrastructure or a cloud architecture, such as using a cloud infrastructure provided by Amazon Web Services™ (AWS), Microsoft™ Azure™, Google Cloud Platform™ (GCP), or Oracle™ Cloud Infrastructure (OCI).

The communication management system 132 may permit the dosing management system 130 to communicate over the network 120 with the clinician monitoring device 110. The communication management system 132 can include an application programming interface (API), such as a cloud API, to facilitate its communications.

The data processing system 134 can process input data (for example, [i] measured values of one or more physiological parameters for the patient, [ii] past dosing amounts or timings for the patient, [iii] laboratory test data for the patient, [iv] dialysis data for the patient, [v] data which may not change or changes minimally over the considered time frame, like demographic data, time on dialysis, diabetic status, weight, gender, or age of the patient, or [vi] planned or potential future dosing amounts or timings for the patient) to determine output data, such as predicated values of one or more physiological parameters or dosing recommendations based on the predicted values. The data processing system 134 can use a prediction model 135, which can include one or more neural networks like a recurrent neural network, to predict values of one or more physiological parameters of a patient and provide dosing recommendations based on the predicted values.

The data processing system 134 can implement one or more of the approaches for predicting values of a physiological parameter or determining dosing recommendations described herein. The data processing system 134 can determine that the predicted values reflect a desirable trajectory for the physiological parameter and generate a dosing recommendation to achieve the predicted values (for instance, that matches planned or potential future dosing amounts or timings for the patient that were used as part of the input data to predict the values). The data processing system 134 can vary the input data (such as to adjust the planned or potential future dosing amounts or timings for the patient) to find the desirable trajectory among various dosing scenarios for the patient. The data processing system 134 can use one or more determination rules to determine a dosing recommendation that complies with one or more treatment rules (for example, a maximum permissible dose increase or a target level of the physiological parameter). The dosing recommendation determined by the data processing system 134 can identify one or more potential amounts or one or more potential timings of a drug to be dosed, among other possibilities. In one example, the dosing recommendation can identify a prescriptive dosing amount for a particular drug, such as ESA or etelcalcetide.

The data processing system 134 can influence or control the handling or dispensing of a drug, such as in accordance with the dosing recommendation. For instance, the data processing system 134 can instruct, such as via the network 120, an electronic device (not shown) to dispense an amount of the drug at a certain time or during a certain time window that is consistent with or controlled by the dosing recommendation. The electronic device can be located in a dialysis center or another care setting (such as a patient's home), among other possible locations. The electronic device can moreover automatically dispense the amount of the drug at the certain time or during the certain time window so that, potentially after an authentication requirement is satisfied, a patient or a clinician may timely obtain a therapeutically effective amount of the drug (and may not obtain more or less of the drug).

The data processing system 134 can store or retrieve data (such as the prediction model 135 or the input data) from a data storage 136. The data processing system 134 can moreover train the prediction model 135 to determine predicted values of the physiological parameter from training input data.

The network 120 can be a computer network. Although the network 120 is shown as one connected network, the network 120 can be subdivided into one or more separate networks which may not directly communicate with one another.

Although certain data processing in the computing environment 100 may be described as being performed by the clinician monitoring device 110 or the data processing system 134, the certain data processing can be shifted to a different device or system in the computing environment 100.

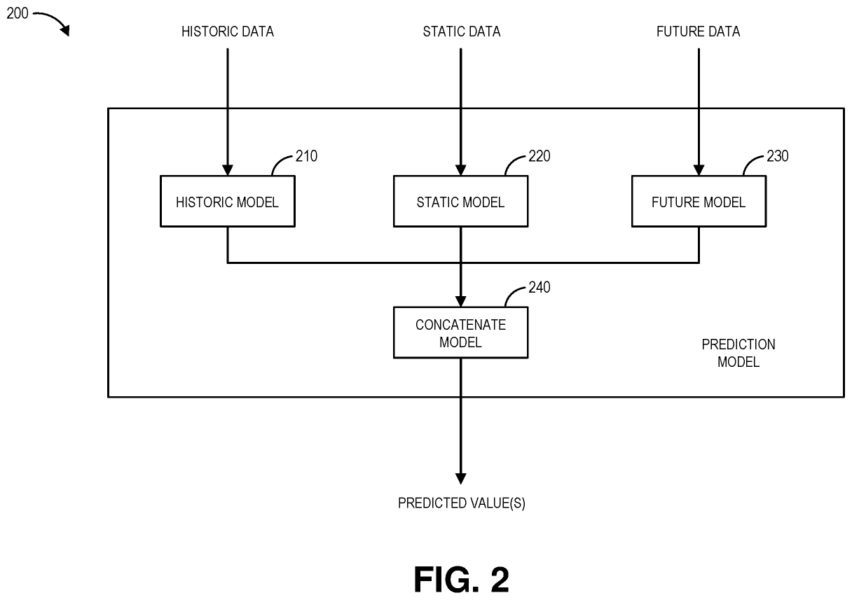

FIG. 2 illustrates a prediction model 200 for a physiological parameter, such as hemoglobin or parathyroid hormone. The prediction model 200 can be an implementation of the prediction model 135 of FIG. 1. The prediction model 200 can include a historic model 210, a static model 220, a future model 230, and a concatenate model 240.

The historic model 210 can receive historic data (for instance, in the form of an input tensor) as an input. The historic data can be indicative of one or more months of a history of a patient, such as (i) measured values of the one or more physiological parameters for the patient, (ii) past dosing amounts or timings for the patient, (iii) laboratory test data for the patient, or (iv) dialysis data for the patient. The historic model 210 can be a recurrent neural network. The historic model 210 can be implemented, for example, using one or more Long Short-Term Memory (LSTM) layers in Keras, as well as potentially one or more Dense layers in Keras. In one implementation, the historic model 210 can include four LSTM layers and one Dense layer arranged in series.

The historic data may be sampled weekly, bi-weekly, or at another frequency, prior to being provided to the historic model 210. The historic data may, for example, be converted using the resample function of Pandas.

In one example, the measured values of the one or more physiological parameters for the patient can be resampled using a weekly frequency. The measured values may be averaged if more than one measured value was measured in a single week. If the measured values of the one or more physiological parameters are not determined weekly and there are one or more weeks where a measured value may not be determined, the historic model 210 can account for this by carrying forward the most recent measured values for up to multiple weeks, such as 5 weeks, (this may be performed because a maximum time between measurements of the values of the one or more physiological parameters may be a particular time period, such as one month) or by setting the measured values to 0.

As another example, for a given laboratory test, the laboratory test time series data for the patient can be resampled using a weekly frequency. The values of the laboratory test time series data can be averaged if more than one test value may be ordered in a single week. The most recent test value of the laboratory test time series data can be carried forward for a variable amount of time (such as depending on how often a test would be expected to be ordered) or set to 0.

As yet another example, dialysis time series data for the patient can be resampled using a weekly frequency. The following measures can be computed for the resulting time series: weekly dialysis count or mean of (a) inter-dialysis weight, (b) blood flow rate, (c) actual dialysis time, (d) patient weight at dialysis, (e) patient Body Mass Index (BMI), or (f) patient Body Surface Area (BSA).

As yet a further example, clinical event times series data (which may, for instance, be indicative of a hospitalization or a blood transfusion) for the patient can be determined. The clinical event times series data can be kept on a periodic basis, such as a weekly basis, to track a number of days that the patient spent in the hospital each week or a number of units of blood transfused each week. The clinical event times series data can then be used to build one or more input tensors with an algorithm. For instance, the algorithm can look for a time window of length t where t may be equal to the sum of a number of months of historic data plus a number of months of future dosing data. The algorithm can check to confirm that (i) there are t+1 consecutive monthly draws of the physiological parameter in the time window and (ii) there are no clinical events that occur after the current monthly draw of the physiological parameter but before the future monthly draw of the physiological parameter. If the checks (i) and (ii) may be satisfied, the time window may be used in one or more input tensors.

As another example, the historic data may be resampled to determine for each week (i) a total dose administered of a drug, (ii) a dosing count indicating a number of times that a drug was dosed, and (iii) a dosing standard deviation indicating a standard deviation of all doses administered. The drug can include erythropoesis stimulating agent, iron, or etelcalcetide, among other possibilities. If no doses were administered or a metric could not be computed for a given week, the resampled value may be replaced with a zero.

Individual features of the historic data can be normalized with respect to characteristics (for instance, a mean or standard deviation) of the historic data or another data set, such as a training data set. For example, a normalized weekly drug data may be generated by dividing drug data by an average patient weight during a given week (for instance, by dividing erythropoesis stimulating agent time series data by the average patient weight for a particular week). The normalized weekly drug data can be converted to a weekly time series representing a percentage change in dose week-on-week.

The static model 220 can receive static data (for instance, in the form of an input tensor) as an input. The static data may not change or changes minimally over the considered time frame. The static data can include, for instance, demographic data, time on dialysis, diabetic status, weight, gender, or age of the patient, among other possibilities. The static model 220 may be a neural network but may, in certain implementations, not be a recurrent neural network. The static model 220 can be implemented, for example, using one or more Dense layers in Keras, such as two Dense layers arranged in series.

The future model 230 can receive future data (for instance, in the form of an input tensor) as an input. The future data can be planned or potential future dosing amounts or timings for the patient of a drug or other compound over a forecast horizon. The future model 230 can be a recurrent neural network. The future model 230 can be implemented, for example, using one or more Long Short-Term Memory (LSTM) layers in Keras, as well as potentially one or more Dense layers in Keras. In one implementation, the future model 230 can include three LSTM layers and one Dense layer arranged in series.

The future data for the future model 230 may be sampled weekly, bi-weekly, or at another frequency prior to being provided to the future model 230. The future data may, for example, be converted using the resample function of Pandas.

In one example, the future data may be resampled to determine for each week (i) a to-be-administered total dose of a drug, (ii) a to-be-administered dosing count indicating a number of times that a drug was dosed, and (iii) a to-be-administered dosing standard deviation indicating a standard deviation of all doses administered. The drug may impact a future level of the physiological parameter for the patient. The drug can, for instance, include erythropoesis stimulating agent, iron, or etelcalcetide, among other possibilities. If no doses were to-be-administered or a metric could not be computed for a given week, the resampled value may be replaced with a zero.

Individual features of the future data can be normalized with respect to characteristics (for instance, a mean or standard deviation) of the future data or another data set, such as a training data set. For example, a normalized weekly drug data may be generated by dividing drug data by an average patient weight during a given week (for instance, by dividing erythropoesis stimulating agent time series data by the average patient weight for a particular week). The normalized weekly drug data can be converted to a weekly time series representing a percentage change in to-be-administered dose week-on-week.

The concatenate model 240 can receive output data from the historic model 210, the static model 220, and the future model 230 (for instance, in the form of output tensors). The concatenate model 240 can process the output data to determine predicted values for the physiological parameter over the forecast horizon. The predicted values can, in some implementations, be output in a set of 1, 2, or 3 month predictions that provide the clinician with a likely trajectory for the patient. The concatenate model 240 can be implemented, for example, using one or more Concatenate layers in Keras.

Where the prediction model 200 may be used for predicting hemoglobin, inputs to the prediction model 200 can include one or more of past measured hemoglobin, past erythropoesis stimulating agent dosing, future potential erythropoesis stimulating agent dosing, past iron dosing, or future iron dosing. Where the prediction model 200 may be used for predicting parathyroid hormone, inputs to the prediction model 200 can include one or more of past measured parathyroid hormone, past etelcalcetide dosing, or future etelcalcetide dosing.

The prediction model 200 can be, for instance, implemented in Python and built using Keras with TensorFlow as a backend. The RMSprop optimizer can be used with Mean Absolute Error as a loss function. The prediction model 200 may, in certain implementations, be run using a batch size of 16 for up to 50 epochs, with an early stopping criterion implemented via the EarlyStopping callback in Keras. In one implementation, the prediction model 200 can receive two months of input data (which may be resampled to a weekly basis) and forecast predicted values for one month (which may be determined on a weekly basis).

Example implementations of the prediction model 200 and associated results of the example implementations are described in the Appendices of U.S. Patent Application Nos. 62/978,687 and 63/066,960. The descriptions of the example implementations and associated results are incorporated by reference herein. As explained in those descriptions, the prediction model 200 is usable to achieve accurate and actionable predicted values for the physiological parameter for at least one, two, or three months in the future.

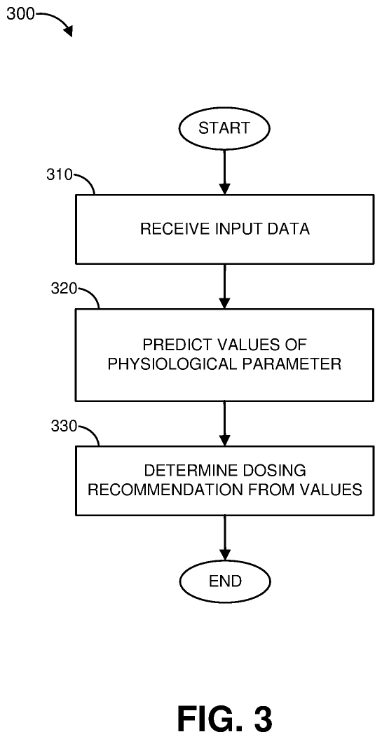

FIG. 3 illustrates a process 300 for determining a dosing recommendation. The process 300 can be implemented by the various components shown in the computing environment 100, such as the data processing system 134. For convenience, the process 300 is described in the context of the computing environment 100, but may instead be implemented by other systems described herein or other computing systems not shown.

At block 310, input data for a patient can be received. For example, the data processing system 134 can receive input data, such as (i) measured values of the one or more physiological parameters for the patient, (ii) past dosing amounts or timings for the patient, (iii) laboratory test data for the patient, (iv) dialysis data for the patient, (v) data which may not change or changes minimally over the considered time frame, or (vi) planned or potential future dosing amounts or timings for the patient of a drug or other compound.

At block 320, values of a physiological parameter can be predicted from the input data. For example, the data processing system 134 can predict the values from the input data using the prediction model 135.

At block 330, a dosing recommendation can be determined from the values. For example, the data processing system 134 can determine that the values reflect a desirable trajectory for the physiological parameter, so the data processing system 134 can generate a dosing recommendation to achieve the values (for instance, that matches planned or potential future dosing amounts or timings for the patient that were used as part of the input data to predict the values at block 320).

### Machine or Computer System Components

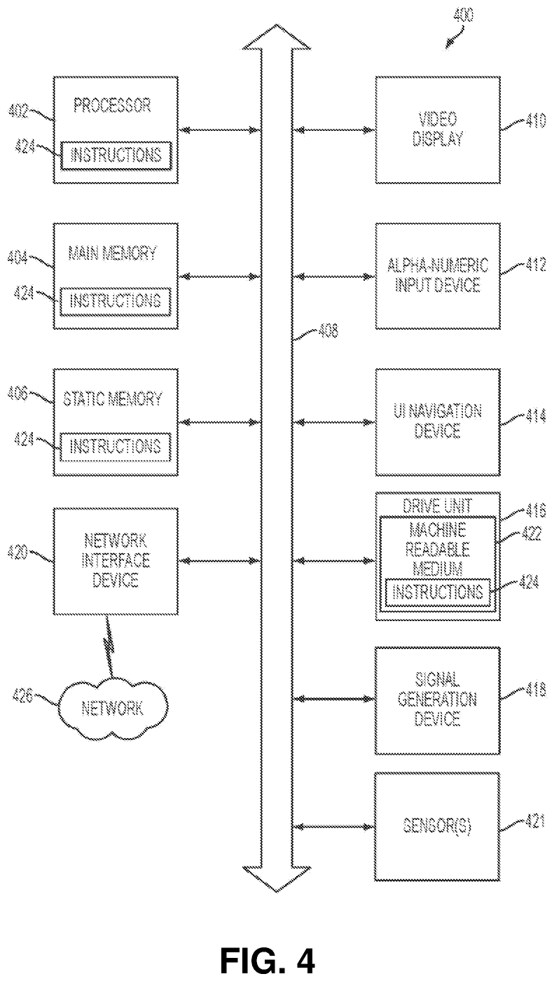

FIG. 4 is a block diagram illustrating a machine 400 upon which one or more features of the present disclosure can be implemented (e.g., run).

Examples of the machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. Where multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs)).

Example apparatus, systems, or methods can be implemented in digital electronic circuitry, in computer hardware, in firmware, in software, or in any combination thereof. Example apparatus, systems, or methods can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

A computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., the machine 400) and software architectures that can be deployed in example embodiments.

In an example, the machine 400 can operate as a standalone device or can be connected (e.g., networked) to other machines.

In a networked deployment, the machine 400 can operate in the capacity of either a server or a client machine in server-client network environments. In an example, the machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while one of the machine 400 is illustrated, the term “machine” shall also be taken to include any collection of machines that individually or jointly execute a set (or multiple sets) of instructions to perform any one or more of the methodologies discussed herein.

The machine 400 can be a computer system and include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417, and UI navigation device 414 can be a touch screen display. The machine 400 can additionally include a storage device 416 (e.g., drive unit), a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include nonvolatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

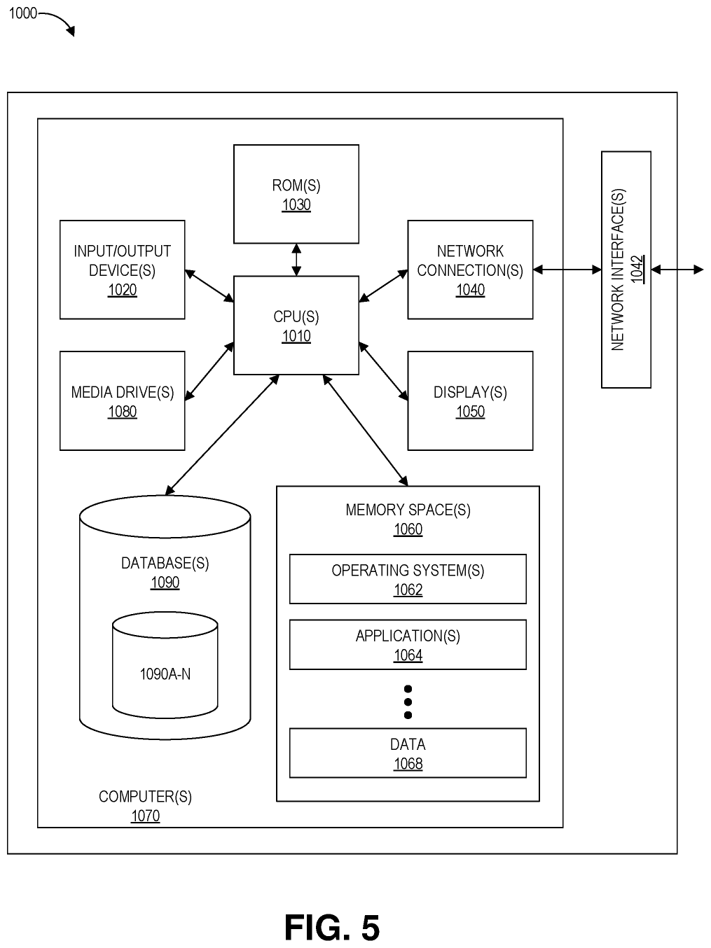

FIG. 5 illustrates a computer system 1000 usable to construct one or more of the devices (for instance, the clinician monitoring device 110), systems (for instance, the dosing management system 130), servers, or the like within the computing environment 100 of FIG. 1.

As shown in FIG. 5, the computer system 1000 can include (i) a processor(s) (CPUs) 1010, (ii) an input/output device(s) 1020 configured to allow users to input and output information and interact with the computer system 1000 as well as transfer and receive data or capture data with one or more sensors like an image sensor, (iii) a read only memory device(s) (ROMs) 1030 or equivalents to provide nonvolatile storage of data or programs, (iv) a display(s) 1050 such as a computer monitor or other display device, (v) a network connection(s) 1040 and a network interface(s) 1042 configured to allow the computer system 1000 to connect to other systems, servers, or portable devices, as well as a memory space(s) 1060 and a database(s) 1090. The database(s) 1090 may be further divided or distributed as sub-database(s) 1090A-1090N, with the sub-database(s) storing feature or function specific information associated with a particular feature or function. The various components shown in FIG. 5 may be incorporated in a computer(s) 1070. It is noted that the various components shown in FIG. 5, including the database(s) 1090, are typically included as part of the computer(s) 1070, however, they may be external to the computer(s) 1070 in some embodiments. For example, the database(s) 1090 may be external to the computer(s) 1070 and may be part of a separate database computer system or networked database system. In some instances, the computer system 1000 may be a computing device like a desktop computer, mobile phone, or a server.

The memory space(s) 1060 may include DRAM, SRAM, FLASH, hard disk drives, or other memory storage devices, such as a media drive(s) 1080, configured to store an operating system(s) 1062, an application program(s) 1064, and data 1068, and the memory space(s) 1060 may be shared with, distributed with or overlap with the memory storage capacity of the database(s) 1090. In some embodiments, the memory space(s) 1060 may include the database(s) 1090, or in some embodiments, the database(s) 1090 may include the data 1068 as shown in the memory space(s) 1060. The data stored in the memory space(s) 1060 or the database(s) 1090 may include information, such as measurement data or data processing routines, or other types of data described herein.

### Other Variations and Terminology

As used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

“Comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing examples, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure.

Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5).

Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The various illustrative logical blocks, modules, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, a microprocessor, a state machine, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A hardware processor can include electrical circuitry or digital logic circuitry configured to process computer-executable instructions. In another embodiment, a processor includes an FPGA or other programmable device that performs logic operations without processing computer-executable instructions. A processor can also be implemented as a combination of computing devices, e.g., a combination of a DSP and a microprocessor, a plurality of microprocessors, one or more microprocessors in conjunction with a DSP core, or any other such configuration. A computing environment can include any type of computer system, including, but not limited to, a computer system based on a microprocessor, a mainframe computer, a digital signal processor, a portable computing device, a device controller, or a computational engine within an appliance, to name a few.

The steps of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC.

Conditional language used herein, such as, among others, “can,” “might,” “may,” “e.g.,” and the like, unless specifically stated otherwise, or otherwise understood within the context as used, is generally intended to convey that certain aspects include, while other aspects do not include, certain features, elements or states. Thus, such conditional language is not generally intended to imply that features, elements or states are in any way required for one or more aspects or that one or more aspects necessarily include logic for deciding, with or without author input or prompting, whether these features, elements or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

What is claimed is:

1. A system for predicting values of a physiological parameter for a patient to determine and output a dosing recommendation for a drug usable to influence future measurements of the physiological parameter, the system comprising:
one or more processors configured to:
receive measurements of a physiological parameter for a patient,
determine, using a plurality of models, values of the physiological parameter from the measurements and future dosing data for the patient, the future dosing data for the patient indicating administration of a drug to the patient to influence future measurements of the physiological parameter,
wherein the plurality of models comprises a first recurrent neural network and a second recurrent neural network different from the first recurrent neural network, the first recurrent neural network being configured to process the measurements for the patient and the second recurrent neural network being configured to process the future dosing data for the patient,
determine a dosing recommendation for the drug from the values, and
output the dosing recommendation for presentation on a display to a caregiver; and
a memory device configured to store the plurality of models.

2. The system of claim 1, wherein the physiological parameter comprises hemoglobin, and the drug comprises erythropoesis stimulating agent.

3. The system of claim 1, wherein the physiological parameter comprises parathyroid hormone, and the drug comprises etelcalcetide.

4. A system for predicting values of a physiological parameter for a patient, the system comprising:
one or more processors configured to:
receive measurements of a physiological parameter for a patient,
determine, using a recurrent neural network, values of the physiological parameter from the measurements for the patient, and
output the values for presentation to a caregiver; and
a memory device configured to store the recurrent neural network.

5. The system of claim 4, wherein the one or more processors are configured to determine the values further from future dosing data for the patient.

6. The system of claim 5, wherein the one or more processors are configured to determine the values using a plurality of recurrent neural networks including the recurrent neural network, the recurrent neural network being configured to process the measurements for the patient and another of the plurality of recurrent neural networks being configured to process the future dosing data for the patient.

7. The system of claim 6, wherein the future dosing data for the patient comprises first dosing data for a first dosing regimen and second dosing data for a second dosing regimen different from the first dosing regimen, and a first set of the values indicates estimated measurements of the physiological parameter under the first dosing regimen and a second set of the values indicates estimated measurements of the physiological parameter under the second dosing regimen.

8. The system of claim 7, wherein the one or more processors are configured to indicate that the first dosing regimen is preferred for the patient over the second dosing regimen.

9. The system of claim 6, wherein the one or more processors are configured to resample the measurements and the future dosing data for the patient to a weekly basis prior to determining the values from the measurements and the future dosing data for the patient.

10. The system of claim 6, wherein the values are indicative of estimated measurements of the physiological parameter between one month and three months in the future.

11. The system of claim 6, wherein the future dosing data for the patient is indicative of administration of a drug to the patient to influence future measurements of the physiological parameter.

12. The system of claim 11, wherein the one or more processors are configured to instruct, based at least on the values, an electronic device to dispense an amount of the drug.

13. The system of claim 4, wherein the one or more processors are configured to determine a dosing recommendation for the patient from the values.

14. The system of claim 13, wherein the dosing recommendation indicates a timing and an amount of a drug to be administered to the patient.

15. The system of claim 14, wherein the one or more processors are configured to output the dosing recommendation for presentation to the caregiver.

16. The system of claim 15, wherein the physiological parameter comprises hemoglobin, and the drug comprises erythropoesis stimulating agent.

17. The system of claim 15, wherein the physiological parameter comprises parathyroid hormone, and the drug comprises etelcalcetide.

18. The system of claim 4, wherein the one or more processors are configured to output the values as part of a graph for presentation on a display to the caregiver.

19. The system of claim 4, wherein the physiological parameter comprises hemoglobin or parathyroid hormone.

20. A method for predicting values of a physiological parameter for a patient, the method comprising:
receiving, by one or more processors, measurements of a physiological parameter for a patient;
storing, by a memory device, a recurrent neural network;
using the recurrent neural network, determining, by one or more processors, values of the physiological parameter from the measurements for the patient; and
outputting the values for presentation to a caregiver.
